(19) United States
(12) Patent Application Publication
(10) Pub. No.: US 2006/0009727 A1
(43) Pub. Date: Jan. 12, 2006

(54) Method and apparatus for an extracorporeal control of blood glucose

(75) Inventors: John J. O'Mahony, Barna (IE); Donald W. Landry, New York, NY (US)

Correspondence Address: NIXON & VANDERHYE, PC, 901 NORTH GLEBE ROAD, 11TH FLOOR, ARLINGTON, VA, 22203, US

(73) Assignee: CHF Solutions Inc., Brooklyn Park, MN (US)

(21) Appl. No.: 11/101,439

(22) Filed: Apr. 8, 2005

Related U.S. Application Data
(60) Provisional application No. 60/560,354, filed on Apr. 8, 2004.

(51) Int. Cl. A61M 37/00; A61M 31/00

(52) U.S. Cl. 604/4.01; 604/65; 604/66

(57) ABSTRACT

A method for controlling the blood glucose level of a patient and periodically calibrating the glucose sensor using a calibration solution. The method controls the level of blood glucose in a patient through an extracorporeal blood circuit by: withdrawing blood from a vascular system in the patient to the extracorporeal circuit; removing ultrafiltrate from the withdrawn blood in the circuit; determining a level of glucose present in the blood based on the removed ultrafiltrate; infusing at least a portion of the removed ultrafiltrate and the withdrawn blood into the vascular system, and infusing insulin into the patient based on the determined level of glucose.

### FIELD OF INVENTION

The invention relates to the field of controllers for controlling the level of glucose in a patient's body. The invention is particularly suitable for the treatment of hyperglycemia and insulin resistance in critically ill patients even those who have not previously been diabetic in the hospital setting.

### BACKGROUND OF THE INVENTION

Critically ill patients that require intensive care for more than five days have a 20% risk of death and substantial morbidity. Hyperglycemia associated with insulin resistance is common in critically ill patients, even those who do not suffer from diabetes. A recent paper published in November 2003 in the NEJM by Greet Van den Burghe et al hypothesized that hyperglycemia or relative insulin deficiency during critical illness may directly or indirectly confer a predisposition to complications such as severe infections, polyneuropathy, multiple-organ failure, and death. In nondiabetic patients with protracted critical illnesses, high serum levels of insulin-like growth factor-binding protein 1, which reflect an impaired response of hepatocytes to insulin, increase the risk of death. They performed a prospective, randomized, controlled trial at one center to determine whether normalization of blood glucose with intensive insulin therapy reduces mortality and morbidity among the critically ill patients.

Van Den Berghe et al were able to show dramatic improvements in patient's outcomes when patients had their blood glucose controlled tightly between 80 and 110 mg per deciliter during their ICU stay.

The trial performed was a prospective, randomized, controlled study involving adults admitted to the surgical intensive care unit who were receiving mechanical ventilation. On admission, patients were randomly assigned to receive intensive insulin therapy (maintenance of blood glucose at a level between 80 and 110 mg per deciliter [4.4 and 6.1 mmol per liter]) or conventional treatment (infusion of insulin only if the blood glucose level exceeded 215 mg per deciliter [11.9 mmol per liter] and maintenance of glucose at a level between 180 and 200 mg per deciliter [10.0 and 11.1 mmol per liter]).

At 12 months, with a total of 1,548 patients enrolled, intensive insulin therapy reduced mortality during intensive care from 8.0 percent with conventional treatment to 4.6 percent (P<0.04, with adjustment for sequential analyses). The benefit of intensive insulin therapy was attributable to its effect on mortality among patients who remained in the intensive care unit for more than five days (20.2 percent with conventional treatment, as compared with 10.6 percent with intensive insulin therapy, P=0.005). The greatest reduction in mortality involved deaths due to multiple-organ failure with a proven septic focus. Intensive insulin therapy also reduced overall in-hospital mortality by 34 percent, bloodstream infections by 46 percent, acute renal failure requiring dialysis or hemofiltration by 41 percent, the median number of red-cell transfusions by 50 percent, and critical-illness polyneuropathy by 44 percent. Also patients receiving intensive therapy were less likely to require prolonged mechanical ventilation and intensive care.

Intensive insulin therapy to maintain blood glucose at or below 110 mg per deciliter was shown to reduce morbidity and mortality among critically ill patients in the surgical intensive care unit. These results are even more exciting when overlaid with Oye et al. (Chest 99:685,1991) findings that 8% of patients consumed 50% of cumulative ICU resources (measured by TISS points) (Therapeutic Intervention Scoring System). Garland et al. (AJRCCM 157:A302, 1998) had similar findings; 5% with the longest ICU lengths of stay consumed 20-48% of various ICU resources.

In the intensive treatment group, an insulin infusion was started if the blood glucose level exceeded 110 mg/dl, adjustment of insulin does was based upon whole-blood glucose measurements in arterial blood at 1 to 4 hour intervals with the use of a blood glucose analyzer. The dose of insulin was adjusted based upon a predetermined algorithm by a team if ICU nurses assisted by a study physician. These manual methods were extremely labor intensive and are not feasible for therapy adoption. In the conventional treatment group a continuous infusion of insulin was started if the blood glucose level exceeded 215 mg/dl and the infusion was adjusted to maintain a level between 180 and 200 mg/dl. On admission all patients were continuously with intravenous glucose (200 to 300 grams per 24 hrs). The next day total parenteral, combined parenteral and enteral feeding was instituted.

Diabetes companies are currently focused on implementing closed loop control for ambulatory diabetic patients where they have encountered a myriad of problems associated with blood glucose sensor accuracy and glucose level control due to the large fluctuations in patient metabolism and eating patterns, changes in sensor sensitivity due to the elapse of time and differences in patients, safety detection systems etc. Much research work is currently being focused to commercially produce an accurate long term implanted blood glucose sensor. It has been found that ensuring blood glucose sensor accuracy and having a fast responsive time are mutually exclusive for an implantable blood glucose sensor. Some glucose sensor manufacturers have focused on subcutaneous implanted sensors to avoid the pitfalls of sensor degradation due to fouling and clotting but these devices, while avoiding the need for blood contact, suffer from longer time constants and transport delays that make closed loop control very difficult. Non-invasive optical methods using near-infrared spectroscopy suffer from the affects of tissue variation and some manufacturers require the use of individual patient calibration making their use less desirable. Other sensors extract glucose through the skin by iontophoresis and measures the extracted sample electrochemically, using the glucose oxidase reaction. Direct contact with blood has been avoided due to clotting and fouling issues.

Thevenot in 1982 (Diabetes Care, Vol. 5 No. 3:184-189) recognized in his article that an implanted sensor would have to survive long-duration implantation in chemically harsh environment of the body. That the sensitivity would have 2 to 5% of the actual glucose level with a range of 10 to 200 mg/dl with little or no change due to long term drift or temperature dependence. Oberhardt in 1982 (Diabetes Care, Vol. 5 No. 3:213-217) recommended that the response of the sensor be 30 sec or less and that the sampling rate be 10 sec averaged over a 1 minute interval. No glucose has yet been proven to meet these requirements.

Many of the design constraints imposed by the ambulatory market are not valid for inpatient hospital ICU use and thus afford a new look at the design requirements. ICU patients are not ambulatory diabetic patients and are fed both parenterally and entrally. This avoids the large swings in levels of blood glucose seen in diabetic patients due to calorie intake at meal times and makes for a more even and predictable control system. Avoiding these large perturbations to the control system makes it easier to maintain glucose control. Implanted glucose sensors would be expected to work accurately for at least one year. This imposes a very large burden upon the sensor design which is currently one the biggest limitation in developing a viable implanted system. If the calibration of such a sensor were to fail it could have deleterious consequences for the patients. Schemes have been proposed to cross check the readings between the implanted sensor and standard finger stick sensors to overcome some of these limitations. Such a limitation does not exist if the sensor is only required for 3 to 5 days of use and independent periodic calibration can be instituted off line ensuring the accuracy of the sensor.

There is a significant need for an easy to use accurate glucose control therapy that can be instituted safely and effectively in the inpatient hospital setting in post surgical ICU patients. Such a therapy will reduce the incidence of mortality, sepsis and renal failure and can have dramatic costs savings for both hospitals and health care providers while improving patient quality of life and outcomes.

### SUMMARY OF THE INVENTION

A device has been developed for controlling the level of glucose in critically ill patients in a hospital that does not suffer from the limitations of currently proposed implanted closed loop control devices. The majority of ICU patients have a short term CVC (Central Venous Catheters) implanted shortly after admission for the purpose of taking clinical measurements and infusing drug therapies. Such catheters are generally between 7 and 8F (French), double or triple lumen and can support blood flows of 40 ml/min or less. Such catheters are ideal of low flow extracorporeal therapy because they can sustain low flow for extended periods of time (>72 hrs) with few interruptions without clotting or causing access issues. This has been the experience of the Aquadex® System 100 fluid removal device in the ICU environment.

An extracorporeal circuit is used to withdraw and infuse blood from and to a patient while simultaneously removing ultrafiltrate in order to overcome the known issues with the calibration, sensitivity and reliability associated with implanted glucose sensors. Blood is withdrawn from the patient with a blood pump, pumped through a filter before being infused back into the patient. In one embodiment, blood is withdrawn from the patient, ultrafiltrate is removed from the blood as it passes through the filter and the ultrafiltrate is pumped by a glucose sensor before being returned with the filtered blood to the patient. An insulin pump is used to infuse insulin into the return blood of the patient as a function of the previous and current ultrafiltrate glucose reading.

In another embodiment, the glucose sensor is periodically calibrated with a known concentration solution of glucose. The ultrafiltrate line can be periodically switched from extracting ultrafiltrate to extracting a calibration solution via a valve system. The valve can be toggled electrically, manually or by the direction of the pump rotation to initiate a calibration. Size not being a limitation, a check and balance system can be more easily implemented to improve sensor accuracy and patent safety. Over 10% of post surgical patients suffer from fluid overload and having a device that can both control blood glucose and remove excess fluid offers a number of advantages to the clinician. It minimizes the number of access sites required by the patient while allowing the clinician to stabilize the patient and treat the underlying condition of the disease.

In another embodiment, the ultrafiltrate is returned upstream of the filter, facilitating the predilution of the filter with ultrafiltrate and reducing the filters propensity to clot. This will have the effect of increasing the response time of the glucose measurement because a certain percentage of old glucose sample will be entrained with the new blood entering the filter. Since the volume of the filter is very small in respect to the blood flow rate this delay is inconsequential.

In theory it is not necessary to perform ultrafiltration to transport glucose across the membrane, diffusion will also transport glucose across the permeable membrane. Diffusion occurs at a much lower rate than convection and would increase the response time of the glucose sensor. The diffusion rate across a membrane is a function of the permeability of the filter and the difference in concentration of the substance in question across the filter and is a derivation of Ficks Law.

A hollow membrane fiber filter is used to separate plasma water from blood for the purposes of removing sensor contaminants such as proteins, albumin, white blood cells and red blood cells from blood which could affect the operation of a blood glucose sensor. Whole blood enters the bundle of hollow fibers from the connector on the top of the cap of the filter canister. Blood flows through a channel approximately 0.2 mm in diameter in each fiber. The walls of the channel are made of a porous material. The pores are permeable to water and small solutes but impermeable to red blood cells, proteins and other blood components that are larger than 40,000-60,000 Daltons. Blood flow in fibers is tangential to the surface of the filter membrane. The shear rate resulting from the blood velocity is high enough such that the pores in the membrane are protected from fouling by particles, allowing the filtrate to permeate the fiber wall. Filtrate (ultrafiltrate) leaves the fiber bundle and is collected in space between the inner wall of the canister and outer walls of the fibers.

The extracorporeal blood controller discriminates between minor difficulties that can be cured automatically and more serious problems that require the attention of a nurse or other medical professional. For example, there is a need for a controller for an extracorporeal blood circuit that can automatically react to partial occlusions in a blood withdrawal or infusion catheter or prompt the patient to move his arm or body to alleviate the occlusion. It may be advantageous for the controller to distinguish between minor difficulties in the blood circuit, such as partial occlusions, and more serious problems, such as total occlusions or extended partial occlusions. For more serious problems, the controller may issue an alarm to a nurse.

A blood withdrawal system has been developed that enables rapid and safe recovery from occlusions in a withdrawal vein without participation of an operator, loss of circuits to clotting, or annoying alarms. The controller may also temporarily stop the blood withdrawal in the presence of a total occlusion and, in certain circumstances, infuses blood into the catheter with a total occlusion. Further, the controller may stop or slow filtration during periods of reduced blood flow through the blood circuits so as to prevent excessive removal of liquids from the blood of a patient. In response to occlusion, blood and ultrafiltrate pump rates are reduced automatically. If occlusion is removed, these flow rates are restored immediately and automatically. The patient is prompted to move, if the occlusion persists for more than a few seconds. The operator is alarmed if occlusions are prolonged or frequent. An alarm is canceled automatically if the occlusion is alleviated, and blood and ultrafiltrate flows are restored. These infusion pressure changes are also monitored by the controller which may adjust the pump flow rate to accommodate such changes.

The glucose controller may be incorporated with a blood withdrawal and infusion pressure control system which optimizes blood flow at or below a preset rate in accordance with a controller algorithm that is determined for each particular make or model of an extraction and infusion extracorporeal blood system. The access controller is further a blood flow control system that uses a real time pressure measurement as a feedback signal that is applied to control the withdrawal and infusion pressures within flow rate and pressure limits that are determined in real time as a function of the flow withdrawn from venous access.

The access controller may govern the pump speed based on control algorithms and in response to pressure signals from pressure sensors that detect pressures in the blood flow at various locations in the extracorporeal circuit. One example of a control algorithm is a linear relationship between a minimum withdrawal pressure and withdrawal blood flow. Another possible control algorithm is a maximum withdrawal flow rate. Similarly, a control algorithm may be specified for the infusion pressure of the blood returned to the patient. In operation, the controller seeks a maximum blood flow rate that satisfies the control algorithms by monitoring the blood pressure in the withdrawal tube (and optionally in the infusion tube) of the blood circuit, and by controlling the flow rate with a variable pump speed. The controller uses the highest anticipated resistance for the circuit and does not adjust flow until this resistance has been exceeded. If the maximum flow rate results in a pressure level outside of the pressure limit for the existing flow rate, the controller responds by reducing the flow rate, such as by reducing the speed of a roller pump, until the pressure in the circuit is no greater than the minimum (or maximum for infusion) variable pressure limit. The controller automatically adjusts the pump speed to regulate the flow rate and the pressure in the circuit. In this manner, the controller maintains the blood pressure in the circuit within both the flow rate limit and the variable pressure limits that have been preprogrammed or entered in the controller.

In normal operation, the access controller causes the pump to drive the blood through the extracorporeal circuit at a set maximum flow rate. In addition, the controller monitors the pressure to ensure that it conforms to the programmed variable pressure vs. flow limit. Each pressure vs. flow limit prescribes a minimum (or maximum) pressure in the withdrawal tube (or infusion tube) as a function of blood flow rate. If the blood pressure falls or rises beyond the pressure limit for a current flow rate, the controller adjusts the blood flow by reducing the pump speed. With the reduced blood flow, the pressure should rise in the withdrawal tube (or fall in the return infusion tube). The access controller may continue to reduce the pump speed, until the pressure conforms to the pressure limit for the then current flow rate.

When the pressure of the adjusted blood flow, e.g., a reduced flow, is no less than (or no greater than) the pressure limit for that new flow rate (as determined by the variable pressure vs. flow condition), the controller maintains the pump speed and operation of the blood circuit at a constant rate. The controller may gradually advance the flow rate in response to an improved access condition, provided that the circuit remains in compliance with the maximum rate and the pressure vs. flow limit.

In another embodiment, a separate glucose sensor is used for controlling the infusion rate of insulin and is cross checked against a second glucose sensor which is intermittently calibrated. This second glucose sensor is called the reference glucose sensor and when not in calibration mode it can in turn be use to recalibrated the control input glucose sensor. This technique has the added advantage of having a continuous line glucose measurement never being interrupted while affording the safety of having a second glucose sensor with periodic calibration.

### SUMMARY OF THE DRAWINGS

A preferred embodiment and best mode of the invention is illustrated in the attached drawings that are described as follows:

FIG. 1 illustrates the treatment of a patient with an ultrafiltration system (an exemplary extracorporeal blood circuit) using a controller to monitor and control the glucose concentration of a patient.

FIG. 2a illustrates the operation and fluid path of the extracorporeal blood circuit shown in FIG. 1 with one way valves for facilitating glucose sensor calibration.

FIG. 2b illustrates the operation and fluid path of the extracorporeal blood circuit shown in FIG. 1 with a three port two-way valve for facilitating glucose sensor calibration.

FIG. 3 is a diagram of the control glucose sensor embedded within the fiber bundle of the filter.

FIGS. 4a to 4d are a series of diagrams shown in plan (4a and 4c) and in cross-section (4b and 4d) to depict the operation of a three port three-way stopcock.

FIGS. 5a to 5c are a series of diagrams depicting the operation of the rotary solenoid.

FIG. 6 is a component diagram of the controller (including controller CPU (central processing unit), monitoring CPU and motor CPU), and of the sensor inputs and actuator outputs that interact with the controller.

FIG. 7 is a schematic diagram of the glucose controller.

FIG. 8 is an illustration of the system response to the partial occlusion of the withdrawal vein in a patient.

FIG. 9 is an illustration of the system response to the complete occlusion and temporary collapse of the withdrawal vein in a patient.

FIG. 10 is a diagram of the filter used on the control glucose sensor for comparison with the reference glucose sensor.

### DETAILED DESCRIPTION OF THE INVENTION

An extracorporeal glucose system and controller has been developed which overcomes many of the limitation of currently proposed glucose control systems by enabling the measurement of the concentration of glucose in blood with little or no delay. This affords a much faster control system while protecting the glucose sensor from contamination by blood and facilitating periodic external calibration.

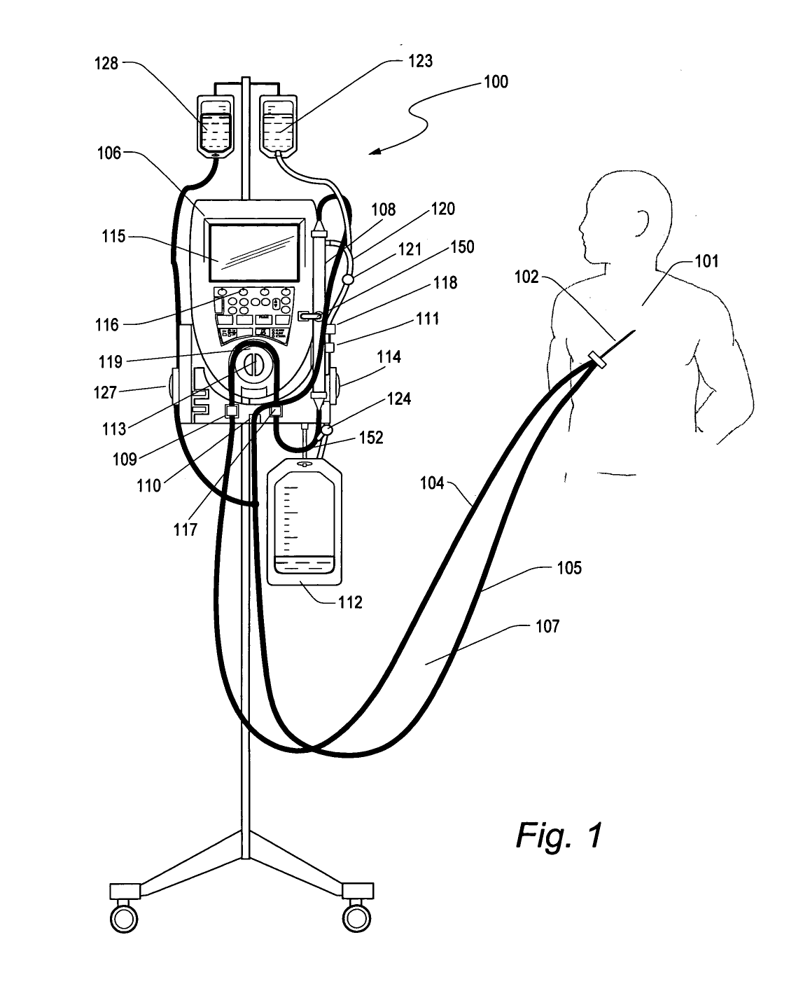

FIG. 1 illustrates the treatment of a patient requiring glucose maintenance with a glucose control apparatus 100. The patient 101, such as a human or other mammal, may be treated while in bed and may be conscious or asleep. The patient need not be confined to an intensive care unit (ICU). To initiate treatment, a standard 7 to 8F, dual or triple lumen CV (central venous) catheter 190 may be used. The catheter is introduced into suitable peripheral or central vein, antecubital, jugular, clavicle or femoral for the withdrawal and return of the blood. The catheter is attached to withdrawal tubing 104 and return tubing 105, respectively. The tubing may be secured to skin with adhesive tape.

The glucose maintenance apparatus includes a blood pump console 106 and a blood circuit 107. The console includes three rotating roller pumps that move blood, ultrafiltrate fluids and insulin through the circuit, and the circuit is mounted on the console. The blood circuit includes a continuous blood passage between the withdrawal line 104 and the return line 105. The blood circuit includes a blood filter 108; pressure sensors 109 (in withdrawal tube), 110 (in return tube) and 111 (in filtrate output tube); an ultrafiltrate collection bag 112 and tubing lines to connect these components and form a continuous blood passage from the withdrawal to the infusion catheters an ultrafiltrate passage from the filter to the ultrafiltrate bag, connections for the attachment of a glucose calibration solution 123 and an insulin infusion bag 128. The ultrafiltrate line 120 is connected to the glucose calibration solution 123 via the tubing 124 by a valve system facilitating the calibration sequence.

The blood passage through the circuit is preferably continuous, smooth and free of stagnate blood pools and air/blood interfaces. These passages with continuous airless blood flow reduce the damping of pressure signals by the system and allows for a higher frequency response pressure controller, which enables the pressure controller to adjust the pump velocity more quickly to changes in pressure, thereby maintaining accurate pressure control without causing instability in control. The components of the circuit may be selected to provide smooth and continuous blood passages, such as a long, slender cylindrical filter chamber, and pressure sensors having cylindrical flow passage with electronic sensors embedded in a wall of the passage. The circuit may come in a sterile package and is intended that each circuit be used for a single treatment.

The circuit mounts on the blood, insulin and ultrafiltrate pumps 113 (for blood passage) 127 for the insulin passage and 114 (for filtrate output of filter). The circuit can be mounted, primed and prepared for operation within minutes by one operator. The operator of the glucose control apparatus 100, e.g., a nurse or medical technician, sets the maximum rate at which fluid is to be removed from the blood of the patient. These settings are entered into the blood pump console 106 using the user interface, which may include a display 115 and control panel 116 with control keys for entering maximum flow rate and other controller settings. Information to assist the user in priming, setup and operation is displayed on the LCD (liquid crystal display) 115. The operator also sets the target glucose level along with upper and lower control limits whereby the console 100 annunciates an alarm when exceeded.

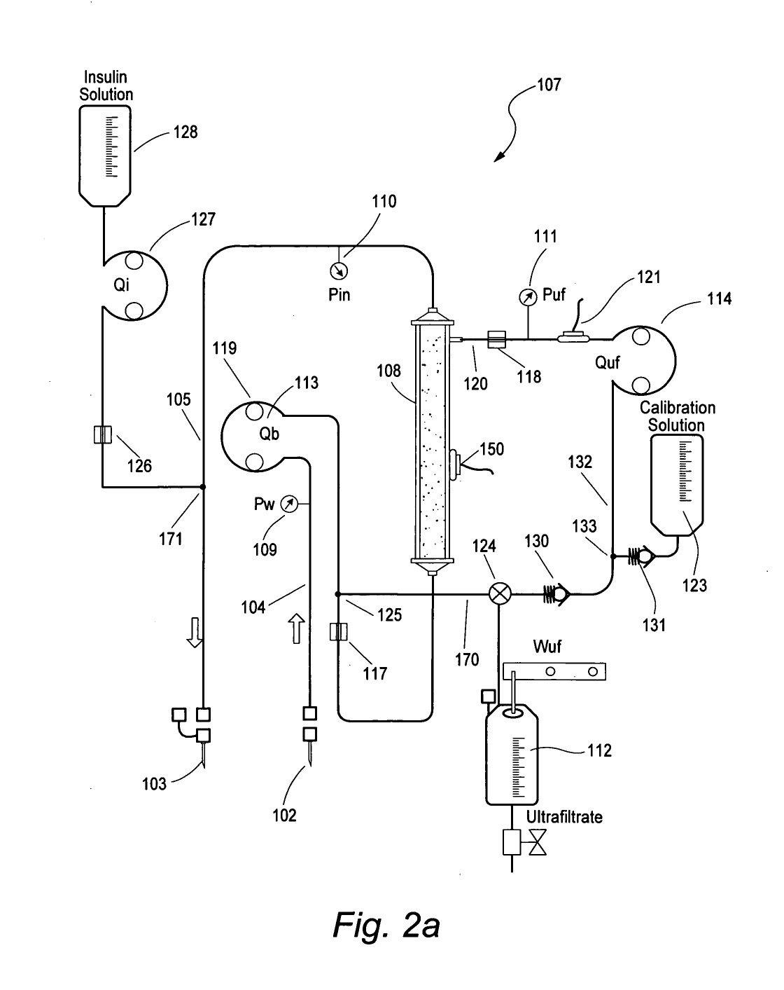

The ultrafiltrate is withdrawn by the ultrafiltrate pump 114 into a graduated collection bag 112 or is returned at the outlet of the blood pump 152 to facilitate predilution of the blood before entering the filter housing 108. The valve 124 may be manually switched by the operator or controlled automatically via a rotary solenoid valve based upon. When the bag is full, ultrafiltration delivery into the bag stops until the bag is emptied. The valve 124 can redirect the ultrafiltrate liquid exiting the ultrafiltrate pump 114 enter the blood line exiting the blood pump and predilute the blood entering the filter 108. The controller may determine when the bag is filled by determining the amount of filtrate entering the bag based on the volume displacement of the ultrafiltrate pump in the filtrate line and filtrate pump speed, or by receiving a signal indicative of the weight of the collection bag. An air detector 117 monitors for the presence of air in the blood circuit, blood is pumped through the circuit. The predilution ultrafiltrate may be returned upstream of the filter and the air detector 117 to ensure that air is not infused into the patient. A blood glucose sensor 150 is connected directly to the filtrate side of the filter with the sensor inserted between the hollow membrane fiber bundles ensuring the fastest signal response possible. A second blood glucose sensor 121 is attached to ultrafiltrate line 120 and can be calibrated with the glucose calibration solution from the bag 123 when the ultrafiltrate pump 114 is reversed via a one way valve 131 (FIG. 2a). A blood leak detector 118 in the ultrafiltrate output line 120 monitors for the presence of a ruptured filter. Signals from the air detector and/or blood leak detector may be transmitted to the controller, which in turn issues an alarm if a blood leak or air is detected in the ultrafiltrate or blood tubing passages of the extracorporeal circuit.

FIG. 2a illustrates the operation and fluid paths of blood, insulin and ultrafiltrate through the blood circuit 107. Blood is withdrawn from the patient through the lumens 102 and 103. The catheter is inserted into a suitable vein defined by current medical practice which can sustain a blood flow of 5 to 40 ml/min. The blood flow from the withdrawal tubing 104 is dependent on the fluid pressure in that tubing which is controlled by a roller pump 113 on the console 106. The algorithms for controlling the withdrawal, infusion and ultrafiltrate pressures are disclosed in U.S. Pat. Nos. 6,796,955; 6,689,083 and 6,706,007 and are incorporated by reference herein.

The length of withdrawal tubing between the withdrawal catheter and pump 113 may be approximately two meters. The withdrawal tubing and the other tubing in the blood circuit may be formed of medical PVC (polyvinyl chloride) of the kind typically used for IV (intravenous) lines which generally has an internal diameter (ID) of 3.2 mm or smaller minimizing blood volume. IV line tubing may form most of the blood passage through the blood circuit and have a generally constant ID throughout the passage.

The pressure sensors may also have a blood passage that is contiguous with the passages through the tubing and the ID of the passage in the sensors may be similar to the ID in the tubing. It is preferable that the entire blood passage through the blood circuit (from the withdrawal catheter to the return catheter) have substantially the same diameter (with the possible exception of the filter) so that the blood flow velocity is substantially uniform and constant through the circuit. Tapered or funnel tubing may be used for the purposes of reducing tubing volume. These tapers occur over such a large length that they do not create dead zones to flow within the tubing. A benefit of a blood circuit having a substantially uniform ID and substantially continuous flow passages is that the blood tends to flow uniformly through the circuit, and does not form stagnant pools within the circuit where clotting may occur.

The roller blood pump 113 is rotated by a brushless DC motor housed within the console 106. The pump includes a rotating mechanism with orbiting rollers that are applied to a half-loop 119 in the blood passage tubing of the blood circuit. The orbital movement of the rollers applied to tubing forces blood to move through the circuit. This half-loop segment may have the same ID as does the other blood tubing portions of the blood circuit. The pump may displace approximately 1 ml (milliliter) of blood through the circuit for each full orbit of the rollers. If the orbital speed of the pump is 60 RPM (revolutions per minute), then the blood circuit may withdraw 60 ml/min of blood, filter the blood and return it to the patient. The speed of the blood pump 113 may be adjusted by the controller to be fully occlusive until a pressure limit of 30 psig (pounds per square inch above gravity) is reached. At pressures greater than 30 psig, the pump rollers relieve because the spring force occluding the tube will be exceeded and the pump flow rate will no longer be directly proportional to the motor velocity because the rollers will not be fully occlusive and will be relieving fluid. This safety feature ensures the pump is incapable of producing pressure that could rupture the filter.

The withdrawal pressure sensor 109 is a flow-through type sensor suitable for blood pressure measurements. It is preferable that the sensor have no bubble traps, separation diaphragms or other features included in the sensor that might cause stagnant blood flow and lead to inaccuracies in the pressure measurement. The withdrawal pressure sensor is designed to measure negative (suction) pressure down to −400 mm Hg.

All pressure measurements in the fluid extraction system are referenced to both atmospheric and the static head pressure offsets. The static head pressure offsets arise because of the tubing placement and the pressure sensor height with respect to the patient connection. The withdrawal pressure signal is used by the microprocessor control system to maintain the blood flow from the vein and limit the pressure.

A pressure sensor may be included in the circuit downstream of the blood pumps and upstream of the filter. Blood pressure in the post pump, pre-filter segment of the circuit is determined by the patient's venous pressure, the resistance to flow generated by the infusion catheter 103, resistance of hollow fibers in the filter assembly 108, and the flow resistance of the tubing in the circuit downstream of the blood pump 113. At blood flows (Qb) of 5 to 40 ml/min, in this embodiment, the pump pressure may be generally in a range of 300 to 500 mm Hg depending on the blood flow, condition of the filter, blood viscosity and the conditions in the patient's vein.

The filter 108 is used to:

Ensure that the glucose sensors 150 and 121 are not contaminated and made inoperable by blood components larger than 50,000 daltons.

Ultrafiltrate the blood and decrease the amount of time it takes for the glucose sensor to get an accurate reading of glucose in the blood.

Remove excess fluid from the patient if necessary.

Whole blood enters the filter 108 and passes through a bundle of hollow filter fibers in a filter canister. There may be between 100 to 1000 hollow fibers in the bundle, and each fiber is a filter. In the filter canister, blood flows through an entrance channel to the bundle of fibers and enters the hollow passage of each fiber. Each individual fiber has approximately 0.2 mm internal diameter. The walls of the fibers are made of a porous material. The pores are permeable to water and small solutes, but are impermeable to red blood cells, proteins and other blood components that are larger than 50,000-60,000 Daltons. Blood flows through the fibers tangential to the surface of the fiber filter membrane. The shear rate resulting from the blood velocity is high enough such that the pores in the membrane are protected from fouling by particles, allowing the filtrate to permeate the fiber wall. Filtrate (ultrafiltrate) passes through the pores in the fiber membrane (when the ultrafiltrate pump is rotating), leaves the fiber bundle, and is collected in a filtrate space between the inner wall of the canister and outer walls of the fibers. The volume of the filter that contains the ultrafiltrate has been designed to be as small as possible and still facilitate the manufacturing of the filter. This volume acts to dampen the real time blood glucose measurements by acting as a reservoir for ultrafiltrate. The dampening effect can be calculated as a first order filter with the time constant calculated as:

$$τ = \frac{{Volume}_{filter}}{{Q}_{UF}}$$

where τ is the first order time constant and can be used to determine the response to change in blood glucose, Volumefilter is the volume of the ultrafiltrate compartment of the filter and QUF is the ultrafiltrate flow. It is evident from this equation that to reduce the response time either the volume must be minimized or the ultrafiltrate flow rate has to be maximized. To help reduce this affect, the blood glucose sensor 150 is embedded in the ultrafiltrate compartment of the filter 108 with the sensor measurement site lying within the polysulphone fibers of the filter. The membrane of the filter acts as a restrictor to ultrafiltrate flow. An ultrafiltrate pressure transducer (Puf) 111 is placed in the ultrafiltrate line upstream of the ultrafiltrate roller pump 114. The ultrafiltrate pump 114 is rotated at the prescribed fluid extraction rate which controls the ultrafiltrate flow from the filter. Before entering the ultrafiltrate pump, the ultrafiltrate passes through approximately 10 cm of plastic tubing 120, the blood leak detector 118, the ultrafiltrate pressure transducer (Puf) and the second reference glucose sensor 121. The tubing is made from medical PVC of the kind used for IV lines and has internal diameter (ID) in this case of 3.2 mm. The ultrafiltrate pump 114 is rotated by a brushless DC motor under microprocessor control. The pump tubing segment (compressed by the rollers) has the same ID as the rest of the ultrafiltrate circuit.

The system may move through the filtrate line approximately 1 ml of filtrate for each full rotation of the pump. A pump speed of 1.66 RPM corresponds to a filtrate flow of 1.66 ml/min, which corresponds to 100 ml/hr of fluid extraction. The ultrafiltrate pump 114 is present to be fully occlusive until a pressure limit of 30 psig is reached. The rollers are mounted on compression springs and relieved when the force exerted by the fluid in the circuit exceeds the occlusive pressure of the pump rollers. The circuit may extract 0 to 500 ml/hr of ultrafiltrate in increments of 10 ml/hr for the clinical indication of fluid removal to relieve fluid overload. When the ultrafiltrate pump 114 rotates clockwise the ultrafiltrate is pumped through the tubing segment 132 through a one way valve 130 and through a valve 124 which is capable of directing the ultrafiltrate to the ultrafiltrate bag 112 or to the filter predilution line 170.

In this operational configuration both the control glucose sensor 150 and the reference glucose sensor measure the concentration of glucose in the blood. The reference glucose sensor 121 has an added lag and time delay due to the volume of ultrafiltrate in the filter filtrate cavity and the volume of tubing between the outlet of the filter 120 and the reference glucose sensor 121. To periodically calibrate the reference glucose sensor 121, the ultrafiltrate pump 114 is reversed. When the ultrafiltrate pump 121 is reversed (rotated anticlockwise) the one way valve 130 prevents ultrafiltrate from the ultrafiltrate bag 112 or blood from the output of the blood pump from entering the return ultrafiltrate line 170. At the same time, glucose calibration solution is drawn through a one way valve 131 connected to the ultrafiltrate line 132 at the T-connection 133. The one-way valve 131 opens due to the negative pressure generated by the reversing ultrafiltrate pump 114. The ultrafiltrate pump is only displaced the volume required to flush the ultrafiltrate line 132 and ensure that the reference glucose sensor is reading an uncontaminated reference solution, e.g., the calibration solution 123. The volume of the tubing between the calibration solution 131 and the reference glucose sensor is less than the volume between the reference glucose sensor and the outlet of the ultrafiltrate from the filter 108. This ensures that during reversal the filtrate cavity of the filter 108 is not contaminated with the glucose calibration solution. During the calibration sequence the control glucose sensor 150 relies on diffusion to measure the correct level of glucose in the blood. The sensor 150 provides an uninterrupted signal for control during the calibration sequence.

After the blood passes through the filter 108, it is pumped through a two meter infusion return tube 105 to the infusion needle 103 where it is returned to the patient. The properties of the filter 108 and the infusion needle 103 are selected to assure the desired TMP (Trans Membrane Pressure) of 150 to 250 mm Hg at blood flows of 5 to 40 ml/min where blood has hematocrit of 35 to 48% and a temperature of room temperature (generally 21 to 23° C.) to 37° C. The TMP is the pressure drop across the membrane surface and may be calculated from the pressure difference between the average filter pressure on the blood side and the ultrafiltration pressure on the ultrafiltrate side of the membrane. Thus, TMP=((Inlet Filter Pressure+Outlet Filter Pressure)/2)−Ultrafiltrate Pressure.

Insulin is also infused into the return line of 105 of the blood circuit. The measurements taken from the control glucose sensor 150 are used to calculate the rate of infusion of glucose required to keep the patients glucose between 80 and 110 mg/dl. An insulin solution is withdrawn from the insulin solution bag 128 and pumped through an air detector 126 before being infused into the return line 105 via the T-connector 171. This configuration is shown with a peristaltic pump 127 but could be replaced with an infusion syringe pump. The pump 127 controls the rate of insulin injection. The controlled insulin rate is determined based on the measured glucose level.

The blood leak detector 118 detects the presence of a ruptured/leaking filter, or separation between the blood circuit and the ultrafiltrate circuit. In the presence of a leak, the ultrafiltrate fluid will no longer be clear and transparent because the blood cells normally rejected by the membrane will be allowed to pass. The blood leak detector detects a drop in the transmissibility of the ultrafiltrate line to infrared light and declares the presence of a blood leak.

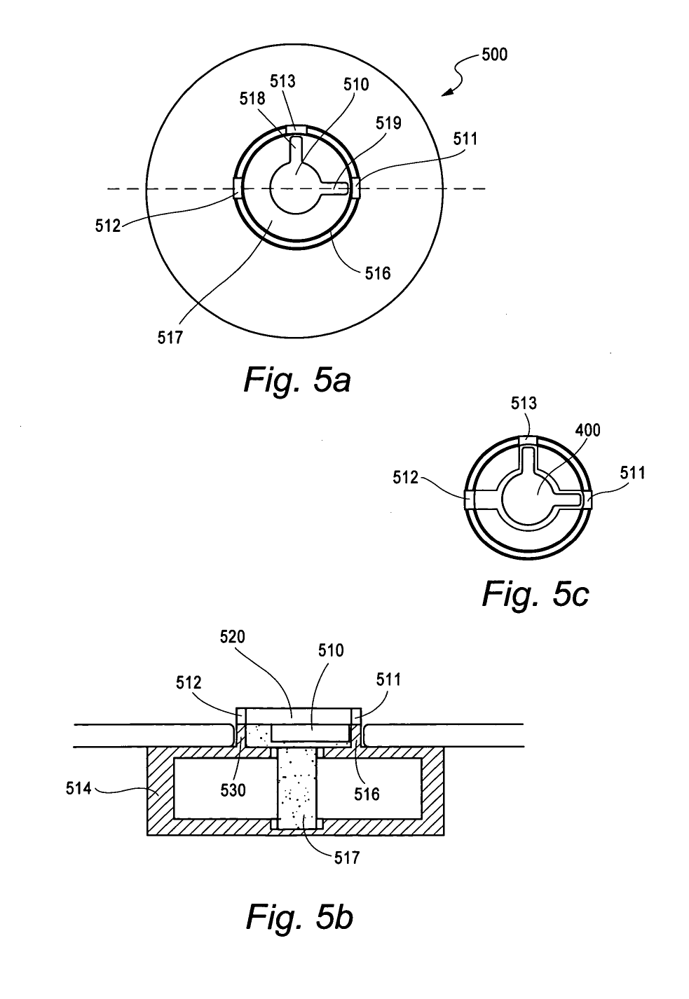

The pressure transducers Pw (withdrawal pressure sensor 109), Pin (infusion pressure sensor 110) and Puf (filtrate pressure sensor 111) produce pressure signals that indicate a relative pressure at each sensor location. Prior to filtration treatment, the sensors are set up by determining appropriate pressure offsets. These offsets are used to determine the static pressure in the blood circuit and ultrafiltrate circuit due to gravity. The offsets are determined with respect to atmospheric pressure when the blood circuit is filled with saline or blood, and the pumps are stopped. The offsets are measures of the static pressure generated by the fluid column in each section, e.g., withdrawal, return line and filtrate tube, of the circuit. During operation of the system, the offsets measured during this static state are subtracted from the raw pressure signals generated by the sensors as blood flows through the circuit. Subtracting the offsets from the raw pressure signals reduces the sensitivity of the system to positional variation between setups and facilitates the accurate measurement of the pressure drops in the circuit due to circuit resistance in the presence of blood and ultrafiltrate flow. Absent these offsets, a false disconnect or occlusion alarm could be issued by the monitor CPU (605 in FIG. 6) because, for example, a static 30 cm column of saline/blood will produce a 22 mm Hg pressure offset.

FIG. 2b illustrates the operation a similar fluid path as that shown in FIG. 2a but in this instance the one way valve system for the infusion of the calibration solution 123 has been replaced with a valve 122 which is capable of switching the flow of fluid to the reference glucose sensor 121 from the output of the ultrafiltrate line 120 to the calibration solution 123. The ultrafiltrate pressure sensor is shown downstream of the valve 122 to ensure maintenance of pressure control limits during calibration. Since the valve and calibration solution lines 124 provide little or no resistance, if the ultrafiltrate pressure is seen to be excessively high when the calibration sequence is in process it is indicative of the calibration solution requiring replenishment or a valve 122 failing to toggle correctly. During calibration, the valve 190 may be toggled to direct the calibration solution to either the ultrafiltrate bag 112 or to the outlet blood line of the blood pump 125. The rest of the fluid path acts in the exact same manner as that outlined in FIG. 2a and is not repeated here.

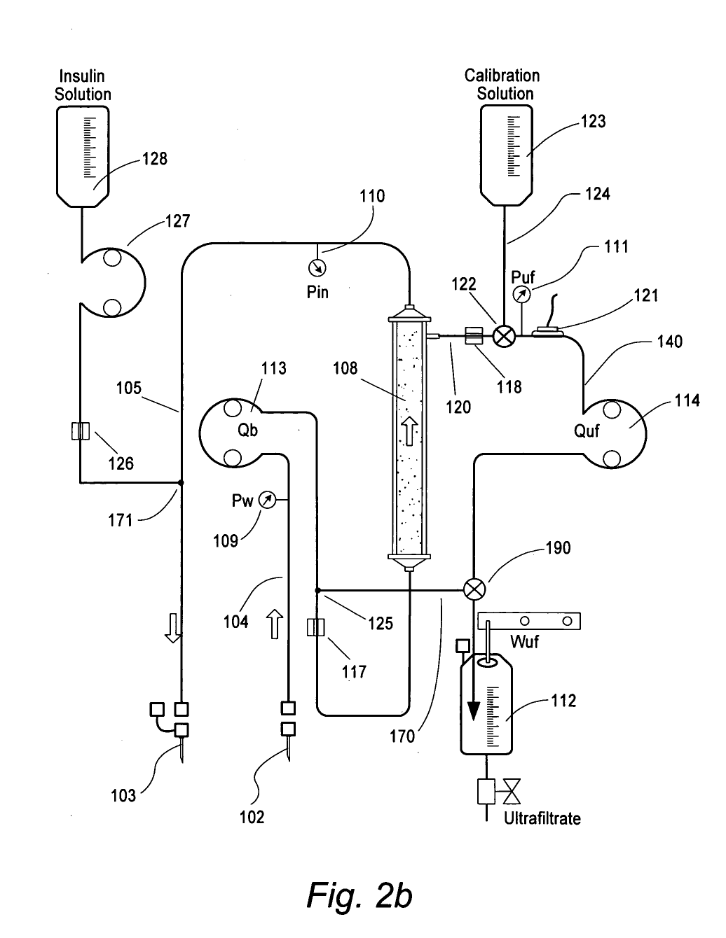

FIG. 3 illustrates the operation and position of the control glucose sensor within the filter fiber bundle. Currently blood glucose sensors are divided into general approaches, electroenzymatic and optical. The electroenzymatic sensors are based upon polarographic principles and utilize the phenomenon of glucose oxidation with a glucose oxidase enzyme. This chemical reaction can be measured electrically by sensing the current output of the sensor. There are two basic optical approaches, infrared absorption spectroscopy and fluorescence based affinity sensors. Any of these sensors can be configured for the approach outlined. As blood 303 passes through the hollow membrane fibers 304 ultrafiltrate is extracted through the permeable wall of the hollow membrane fibers. The sensor 301 is positioned within the fiber bundle to reduce the response time by taking advantage of the diffusion of glucose across the membrane and to minimize the volume of ultrafiltrate that has to be cleared before the control glucose sensor accurately represents the level of glucose in the blood. The control glucose sensor 150 is attached to the wall of the filter canister 306. The ultrafiltrate removed from the blood in the hollow membrane fibers exits the filter canister 306 at the port 302. The filtrate volume represented by 307 in this illustration of the filter canister is minimized to improve signal response time.

One of the most common sensors commercially available for this application is the electrode/oxidation method for determining blood glucose levels. The sensor uses a platinum electrode and a silver electrode to form part of an electric circuit in which hydrogen peroxide is electrolyzed. The hydrogen peroxide is produced as a result of the oxidation of glucose on a glucose oxidase membrane and the current through the circuit provides a measure of the hydrogen peroxide concentration and hence glucose concentration in the vicinity of the sensor. Such a sensor could easily be used for this application.

Optical sensors which use infra red light of two or more wavelengths either transmissively or reflectively are also well suited for this application. Many of the issues with implanting such devices are now overcome, such as sensor size, variations in tissue and individual calibrations for each patient.

The solenoid controlled valve system shown in FIG. 2b can be implemented with standard stopcocks making the valves disposable and enabling them to be components of the disposable blood circuit.

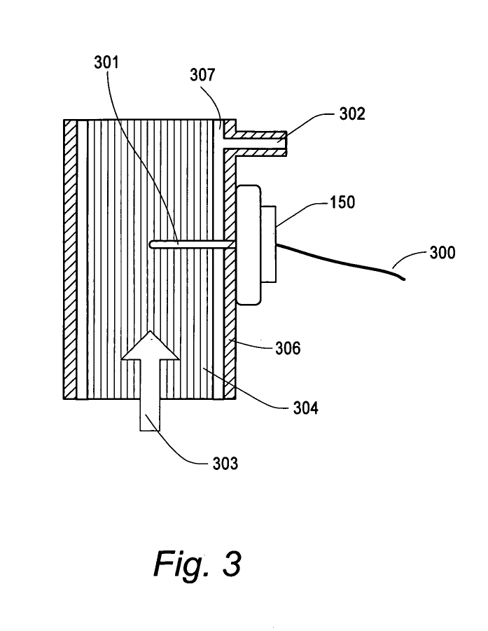

FIG. 4a shows the plan view of a standard three port, two-way stopcock (e.g. Qosina P/N 99743). The stopcock has three ports and can connect two ports together at a time. The lever arm of the stopcock is represented by 410 with arms 403 and 404. The arms point to the ports that are connected 401 and 402. The port 405 is closed in this configuration.

FIG. 4b shows a cross-section of the same valve in the same lever position showing the ports 401 and 402 connected via the conduit 406. The conduit allows fluid to flow from port 401 to 402.

FIG. 4c shows the lever arm 410 rotated 90 degrees anti-clockwise from that displayed in FIG. 4a with the lever arm 404 pointed towards port 401 and lever arm 403 pointed towards port 405. Thus port 401 is the common port and it can be switched from port 402 to port 403 by rotating the lever arm 410 (FIG. 4a)

FIG. 4d shows a cross-section of the valve in the configuration of FIG. 4c with the ports 401 and 405 connected via the conduit 406. The body of the valve 407, swivels as the lever arms are rotated.

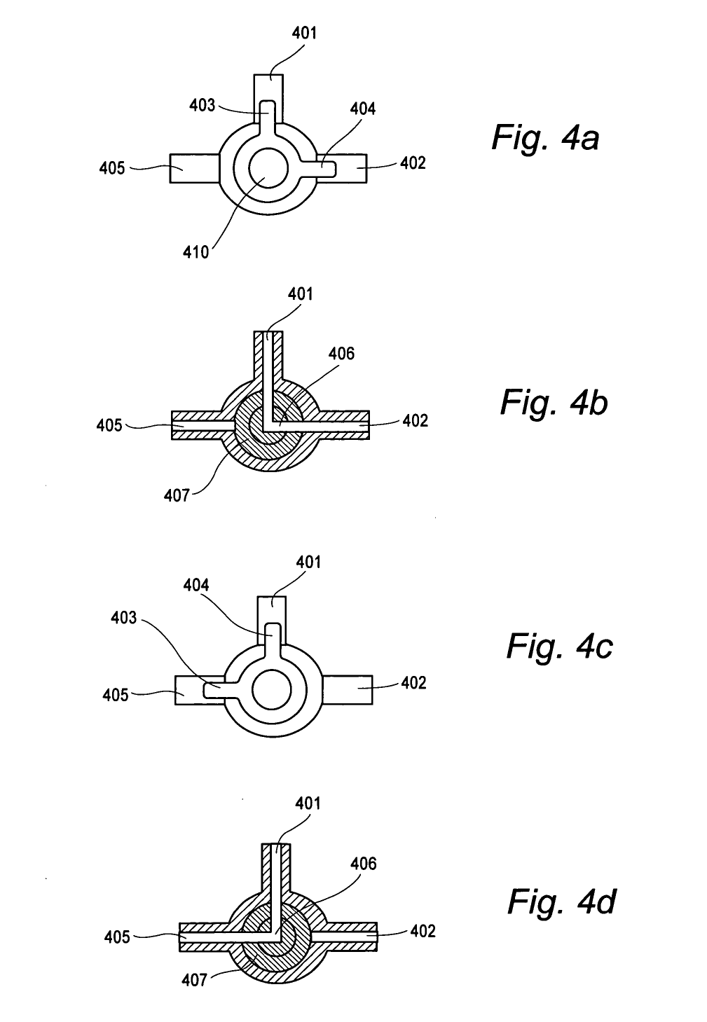

FIGS. 5a, 5b and 5c show a plan and elevation view of a rotary solenoid valve 500 for rotating the stopcock lever arm 410 shown in FIGS. 4a and 4c. The diagram shows how the stopcock 400 (FIG. 4a) fits into a recess in the shaft 520 of the solenoid valve and when rotated redirects flow from ports 401 to 402 to ports 402 to 405 (FIG. 4a). The actuator for rotating the stopcock could also be implemented with a stepper motor or a DC motor. A solenoid valve was chosen for simplicity with a rotation of 90 degrees. During rotating of the solenoid the lever arm of the stopcock is free to move but the body and ports are secured to prevent rotation. The lever arm of the stopcock 400 fits into a machined cavity 510 in the rotational shaft 517 of the solenoid 500. The ports 401, 402 and 405 fit into slotted recesses in the solenoid housing 513, 511 and 512 respectively. This is depicted in greater detail in FIG. 5c. These port recesses cannot rotate because they are connected to the housing 514 of the solenoid whereas the cavity 510 for the lever arm can because it is connected to the shaft 517 of the rotary solenoid. The ports reside in the plane 520 whereas the lever arms reside in the plane 510 shown in FIG. 5b. FIG. 5b also shows how the recesses for the ports 511 and 512 are connected to the main solenoid housing 530. The lever arms 403 and 404 FIG. 4a fit into the recesses of the cavity 510 in the port slots 518 and 519. FIG. 5c depicts an overlay of the stopcock 400 on the shaft of the rotary solenoid valve 500.

The one way valves 130 and 131 in FIG. 2a are spring return valves with a cracking pressure of approximately 1 psi. This prevents leaks due to the static head pressure caused by difference in height between the glucose calibration solution and the position of the one way valve 131 and time delays in the closure of the valve if no back pressure exists.

FIG. 6 illustrates the electrical architecture of the glucose control system 600 (100 in FIG. 1), showing the various signal inputs and actuator outputs to the controller. The user-operator inputs the desired ultrafiltrate extraction rate and the maximum and minimum allowable glucose readings into the controller by pressing buttons on a membrane interface keypad 609 on the controller. These settings may include the maximum flow rate of blood through the system, maximum time for running the circuit to filter the blood, the maximum ultrafiltrate rate and the maximum ultrafiltrate volume. The settings input by the user are stored in a memory 615 (mem.), and read and displayed by the controller CPU 605 (central processing unit, e.g., microprocessor or micro-controller) on the display 610.

The controller CPU regulates the pump speeds by commanding a motor controller 602 to set the rotational speed of the blood pump 113 to a certain speed specified by the controller CPU. The motor controller adjusts the speed of the ultrafiltrate pump 111 in response to commands from the controller CPU and to provide a particular filtrate flow velocity specified by the controller CPU. The motor controller adjusts the speed of the insulin pump 127 in response to commands from the controller CPU and to provide a particular insulin flow velocity specified by the controller CPU. Feedback signals from the pressure transducer sensors 611 and glucose sensors 620 are converted from analog voltage levels to digital signals in an A/D converter 616. The digital pressure signals are provided to the controller CPU as feedback signals and compared to the intended pressure levels and glucose level determined by the CPU. In addition, the blood leak detector, ultrafiltrate weight, pressure signals, motor currents, pump velocities and current blood glucose level are also monitored by an independent monitor CPU 614.

The motor controller 602 controls the velocity, rotational speed of the blood insulin pump and filtrate pump motors 603, 621, 604. Encoders 607, 622, 606 mounted to the rotational shaft of each of the motors as feedback provide quadrature signals, e.g., a pair of identical cyclical digital signals, but 90 o out-of-phase with one another. These signal pairs are fed to a quadrature counter within the motor controller 602 to give both direction and position. The direction is determined by the signal lead of the quadrature signals. The position of the motor is determined by the accumulation of pulse edges. Actual motor velocity is computed by the motor controller as the rate of change of position. The controller calculates a position trajectory that dictates where the motor must be at a given time and the difference between the actual position and the desired position is used as feedback for the motor controller. The motor controller then modulates the percentage of the on time of the PWM signal sent to the one-half 618 bridge circuit to minimize the error. A separate quadrature counter 617 is independently read by the Controller CPU to ensure that the Motor Controller is correctly controlling the velocity of the motor. This is achieved by differentiating the change in position of the motor over time.

The CPU controls the position of the actuation of the rotary solenoid valve 631 via a driver 630. The position of the solenoid valve is determined by feedback from a proximity switch which determines the position of rotary valve via a metal tab. The valve can be actively driven in either direction clockwise or anticlockwise and remains in position due to the latching nature of the rotary solenoid valve. Such valves are supplied by Ledex corporation.

The monitoring CPU 614 provides a safety check that independently monitors each of the critical signals, including signals indicative of blood glucose, blood leaks, pressures in blood circuit, weight of filtrate bag, motor currents, air in blood line detector and motor speed/position. The monitoring CPU has stored in its memory safety and alarm levels for various operating conditions of the glucose control and ultrafiltrate system. By comparing these allowable preset levels to the real-time operating signals, the monitoring CPU can determine whether a safety alarm should be issued, and has the ability to independently stop both motors and reset the motor controller and controller CPU if necessary.

The user can view the level of glucose real time being measured in the ultrafiltrate by examining the LCD display panel of the user setting display 610. Graphs of the glucose level over time may also be selected to view the stability of the control over 1 hr, 4 hr, 8 hr, 24 hr and 72 hr periods. Time periods are selectable via user setting membrane panel 609. In order to provide additional patient safety the user may adjust upper and lower glucose alarm limits or accept the default values of 75 and 120 mg/dL. When the limits are exceeded an audible and visual alarm is annunciated via the speaker 640 and LCD display panel 610 drawing the medical practitioner's attention to a potentially dangerous clinical condition. The LCD displays a message stating the source of the alarm and potential solutions. The purpose of the alarm is to prevent the patient from becoming hypoglycemic or hyperglycemic in the event that the control system fails to maintain the blood glucose level within the desired limits both of which can result in coma and death if left unchecked giving the medical practitioner enough time to intervene and reverse the situation.

The glucose control systems may also be used solely for the purposes of real time monitoring of blood glucose levels. To select this option the active control of glucose may be disabled via the membrane panel 610 ceasing the infusion of insulin. During this mode the user interface via the LCD displays a message to the user that active control of glucose has ceased. In this mode the device can be used to aid the medical practitioner in determining when it is necessary to titrate insulin manually. The alarm limits can be set to highlight when adjustments to manual titration of insulin are necessary obviating the need for the medical practitioner to continuously or intermittently monitor the patient. The monitoring system will alarm if the patients glucose level exceeds preset set alarm limits.

Glucose control systems mimic the body's natural insulin response to blood glucose levels as closely as possible in implanted glucose control applications, because excursions in the body without regard for how much insulin is delivered can cause excessive weight gain, hypertension and atherosclerosis. The same risks are not present in short term ICU care when glucose control is only required for an average of 3 days. In post surgical ventilator dependent patients glucose may be infused at 200 to 300 grams per 24 hr period providing a continuous infusion of glucose and the ability to prevent hypoglycemia when insulin infusion is turned off. When glucose sensors are implanted subcutaneously and the effects of the infusion insulin can have signal delays of up to 30 minutes it can be very difficult to maintain stability especially when the time delay is varying. The proposed system suffers from very little signal time delay and lag. It is not necessary to wait for the insulin to transport through the interstitial space to the blood volume and back again to interstitial space to reach equilibrium. Insulin is infused directly into the blood and is transported directly to the interstitial space and organs. Control is based upon the measurement of the blood glucose level and the only delays and lag which occur are those of the insulin mixing in the blood volume, the transport of blood from the body to the filter and the transport of the ultrafiltrate to the sensor. These delays and lags are extremely short in comparison to those experienced by a subcutaneous glucose sensor. For instance blood is transported to the filter in less than 30 sec (15 ml (circuit volume)/40 ml/min (blood flow)=22.5 sec). Ultrafiltrate is typically removed at 500 ml/hr thus with an ultrafiltrate volume of 10 ml between the sensor and ultrafiltrate exiting the membrane fiber the first order time lag is 1 min 12 sec. Thus the overall delay and response time is well less than 5 minutes.

A measurement delay also exists between the control and reference blood glucose sensors which can be accounted for by taken into account by modeling the plant between the two sensors. Such a model makes the comparison between readings even more accurate and facilitates comparisons during the control of glucose.

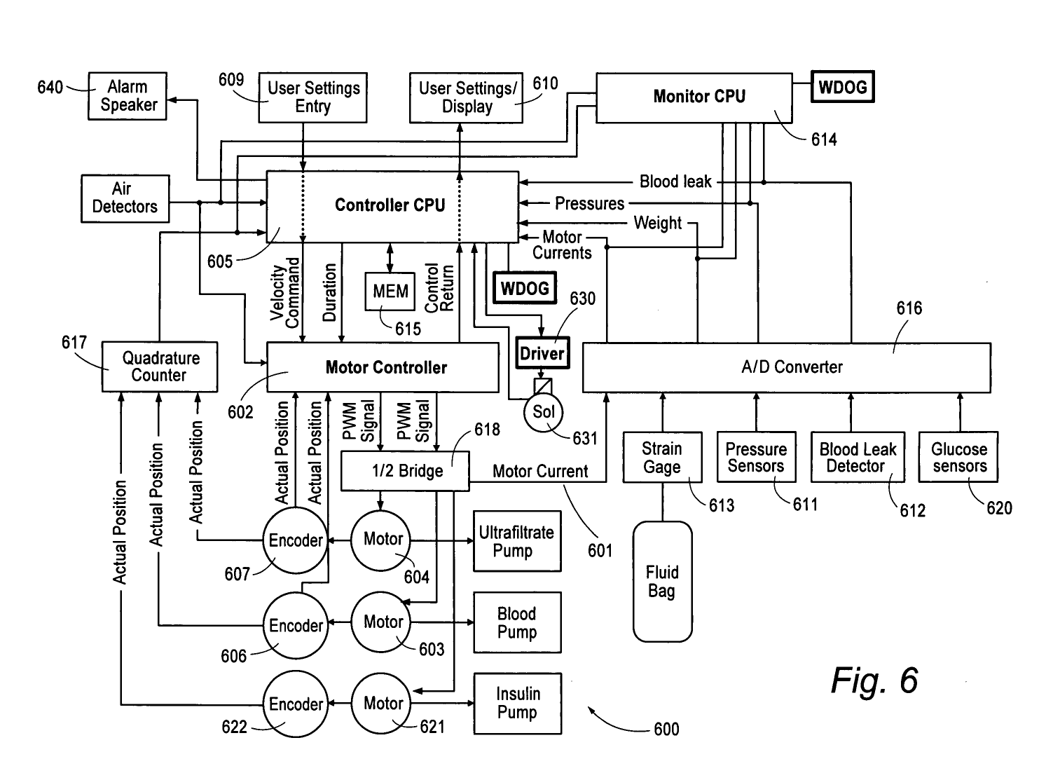

FIG. 7 shows the implementation of a PIDFF (Proportional Integral Derivative Feed Forward) controller whose purpose is to main a target 701 glucose level of the patient of 95 mg/dl. The control glucose sensor is read at a sample rate between 30 seconds and 10 minutes. For the purpose of this explanation it can be assumed that the measurement Gtx 702 is taken every 2 minutes. An error is calculated as Error=Target−Gtx. Based upon this error a proportional 705, integral 706 and determinative term 707 are calculated. The integral term when started for the first time is set to have an output of 2 U/hr of insulin. This is known as a feed forward term and has the function of reducing overshoot. The integral term is limited in both the positive and negative direction to limit windup. In this case the integral has a separate specific minimum integral term allowed minQinlterm. The outputs of the proportional, integral and derivatives are summed and once again limited. For instance the upper and lower limits of the integral term may be +/−10 U/hr whereas the limits of the PIDFF would be limited to +10 U/hr and 0 U/hr because is not possible to deliver a negative insulin dose. But at a particular control point in time the Integral may be −5 U/hr and the proportional term may 6 U/hr thus the total output of the controller would be 1 U/hr assuming the derivative to be 0 U/hr. Such a scheme allows for a more stable control system allowing symmetry in the integral controller. Once the insulin infusion rate is calculated a command is sent to the motor controller to implement the infusion rate.

Since the Glucose control system relies on the withdrawal and infusion of blood, periodic occlusion or partial occlusion may occur which will affect the control system. The withdrawal pressure controller is based upon the withdrawal blood flow but the infusion pressure controller is based upon both the blood flow and the insulin infusion. Since the infusion of insulin is the most important task of the controller it is maintained until the desired blood flow is lower than insulin infusion rate. As the blood flow reduces in response to a partial occlusion the ultrafiltrate rate is reduce not to exceed 20% of the blood flow rate. When the blood flow rate is less than 10 ml/min, 25% of the target blood flow rate of for example 40 ml/min ultrafiltration is stopped and the device alarms to inform the user of the condition. If the set blood flow rate was 5 ml/min then ultrafiltration would be stopped when the blood flow dropped below 1.25 mL/min. Glucose infusion rates are well less than 1 ml/min and in reality have little or no affect on the pressure control. During a total occlusion when the system reverses glucose control is terminated for the duration of the reversal.

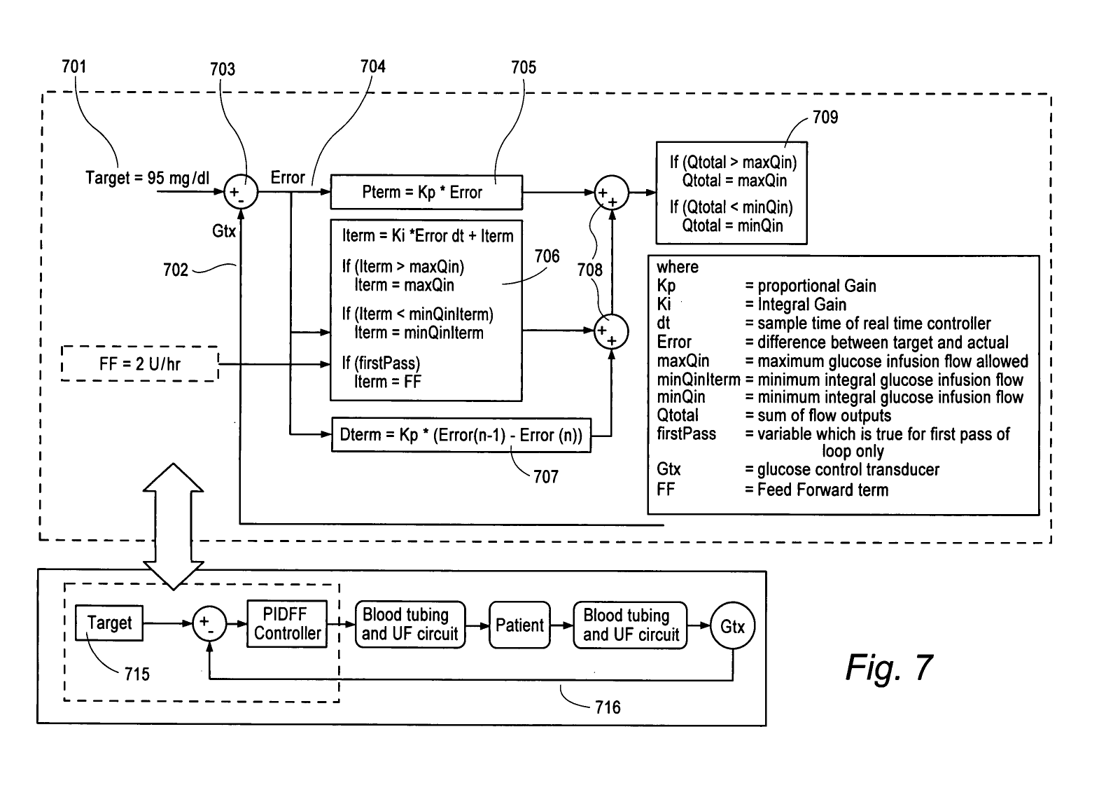

FIG. 8 illustrates the operation of a glucose control device under the conditions of a partial and temporary occlusion of the withdrawal vein. The data depicted in the graph 800 was collected in real time, every 0.1 second, during ultrafiltration treatment of a patient. Blood was withdrawn from the left arm and infused into the right arm in different veins of the patient using similar 18 Gage needles. A short segment of data, i.e., 40 seconds long, is plotted in FIG. 8 for the following traces: blood flow in the extracorporeal circuit 804, infusion pressure occlusion limit 801 calculated by CPU 605 (FIG. 6.0), infusion pressure 809, calculated withdrawal pressure limit 803 and measured withdrawal pressure 802. Blood flow 804 is plotted on the secondary Y-axis 805 scaled in mL/min. All pressures and pressure limits are plotted on the primary Y-axis 806 scaled in mmHg. All traces are plotted in real time on the X-axis 807 scaled in seconds.

In the beginning, between time marks of 700 and 715 seconds, there is no obstruction in either infusion or withdrawal lines. Blood flow 804 is set by the control algorithm to the maximum flow limit of 55 mL/min. Infusion pressure 809 is approximately 150 to 200 mmHg and oscillates with the pulsations generated by the pump. Infusion occlusion limit 801 is calculated based on the measured blood flow of 55 mmHg and is equal to 340 mmHg. Similarly, the withdrawal pressure 802 oscillates between −100 and −150 mmHg safely above the dynamically calculated withdrawal occlusion limit 803 equal to approximately −390 mmHg.

At approximately 715 seconds, a sudden period of partial occlusion 808 occurred. The occlusion is partial because it did not totally stop the blood flow 804, but rather resulted in its significant reduction from 55 mL/min to between 25 and 44 mL/min. The most probable cause of this partial occlusion is that as the patient moved during blood withdrawal. The partial occlusion occurred at the intake opening of the blood withdrawal needle. Slower reduction in flow can also occur due to a slowing in the metabolic requirements of the patient because of a lack of physical activity. Squeezing a patient's arm occasionally will increase blood flow to the arm, which results in a sudden sharp decrease 810 of the withdrawal pressure 802 from −150 mmHg to −390 mmHg at the occlusion detection event 811. The detection occurred when the withdrawal pressure 810 reached the withdrawal limit 803. The controller CPU responded by switching from the maximum flow control to the occlusion limit control for the duration of the partial occlusion 808. Flow control value was dynamically calculated from the occlusion pressure limit 803. That resulted in the overall reduction of blood flow to 25 to 45 mL/min following changing conditions in the circuit.

FIG. 8 illustrates the occlusion of the withdrawal line only. Although the infusion occlusion limit 801 is reduced in proportion to blood flow 804 during the occlusion period 808, the infusion line is never occluded. This can be determined by observing the occlusion pressure 809 always below the occlusion limit 801 by a significant margin, while the withdrawal occlusion limit 803 and the withdrawal pressure 802 intercept and are virtually equal during the period 808 because the PIFF controller is using the withdrawal occlusion limit 803 as a target.

The rapid response of the control algorithm is illustrated by immediate adjustment of flow in response to pressure change in the circuit. This response is possible due to: (a) servo controlled blood pump equipped with a sophisticated local DSP (digital signal processing) controller with high bandwidth, and (b) extremely low compliance of the blood path. The effectiveness of controls is illustrated by the return of the system to the steady state after the occlusion and or flow reduction disappeared at the point 812. Blood flow was never interrupted, alarm and operator intervention were avoided, and the partial occlusion was prevented from escalation into a total occlusion (collapse of the vein) that would have occurred if not for the responsive control based on the withdrawal pressure.

If the system response was not this fast, it is likely that the pump would have continued for some time at the high flow of 55 mL/min. This high flow would have rapidly resulted in total emptying of the vein and caused a much more severe total occlusion. The failure to quickly recover from the total occlusion can result in the treatment time loss, potential alarms emitted from the extracorporeal system, and a potential need to stop treatment altogether, and/or undesired user intervention. Since user intervention can take considerable time, the blood will be stagnant in the circuit for a while. Stagnant blood can be expected to clot over several minutes and make the expensive circuit unusable for further treatment.

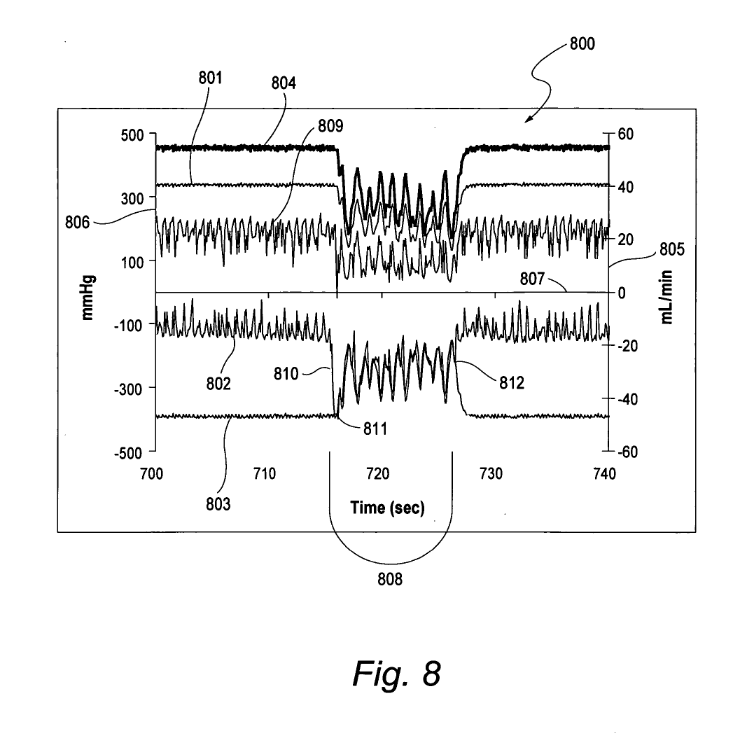

FIG. 9 illustrates a total occlusion of the blood withdrawal vein access in a different patient, but using the same apparatus as used to obtain the data shown in FIG. 8. Traces on the graph 900 are similar to those on the graph 800. The primary Y-axis (months) and secondary Y-axis (mL/min) correspond to pressure and flow, respectively, in the blood circuit. The X-axis is time in seconds. As in FIG. 8 the system is in steady state at the beginning of the graph. The blood flow 804 is controlled by the maximum flow algorithm and is equal to 66 mL/min. The withdrawal pressure 802 is at average of −250 mmHg and safely above the occlusion limit 803 at −400 mmHg until the occlusion event 901. Infusion pressure 809 is at average of 190 mmHg and way below the infusion occlusion limit 801 that is equal to 400 mmHg.

As depicted in FIG. 9, the occlusion of the withdrawal access is abrupt and total. The withdrawal vein has likely collapsed due to the vacuum generated by the needle or the needle opening could have sucked in the wall of the vein. The withdrawal vein is completely closed. Similar to the partial occlusion illustrated by FIG. 8, the rapid reduction of the blood flow 804 by the control system in response to the decreasing (more negative) withdrawal pressure 802 prevented escalation of the occlusion, but resulted in crossing of the occlusion limit 803 into positive values at the point 902. Simultaneously the blood flow 804 dropped to zero and sequentially became negative (reversed direction) for a short duration of time 903. The control system allowed reversed flow continued for 1 second at 10 mL/min as programmed into an algorithm. This resulted in possible re-infusion of 0.16 mL of blood back into the withdrawal vein. These parameters were set for the experiment and may not reflect an optimal combination. The objective of this maneuver is to release the vein wall if it was sucked against the needle orifice. It also facilitated the refilling of the vein if it was collapsed.

During the short period of time when the blood flow in the circuit was reversed, occlusion limits and algorithms in both infusion and withdrawal limbs of the circuit remained active. The polarity of the limits was reversed in response to the reversed direction of flow and corresponding pressure gradients.

The success of the maneuver is illustrated by the following recovery from total occlusion. At the point 904 signifying the end of allowed flow reversal, the withdrawal occlusion limit 803 became negative and the infusion occlusion limit 801 became positive again. The blood pump started the flow increase ramp shown between points 904 and 905. The gradual ramp at a maximum allowed rate is included in the total occlusion recovery algorithm to prevent immediate re-occlusion and to allow the withdrawal vein to refill with blood.

For the example illustrated by FIG. 9, the most likely cause of the occlusion was suction of the blood vessel wall to the withdrawal needle intake opening. The occlusion onset was rapid and the condition disappeared completely after the short reversal of flow that allowed the vessel to re-inflate. It can be observed that while the withdrawal occlusion ramp 907 followed the blood flow ramp 905, the measured withdrawal pressure 906 did not anymore intercept it. In fact, by the time the steady-state condition was restored, the withdrawal pressure 910 was at approximately −160 mmHg. Prior to occlusion the withdrawal pressure level 802 was approximately −200 mmHg. Thus, the withdrawal conditions have improved as a result of the total occlusion maneuver.

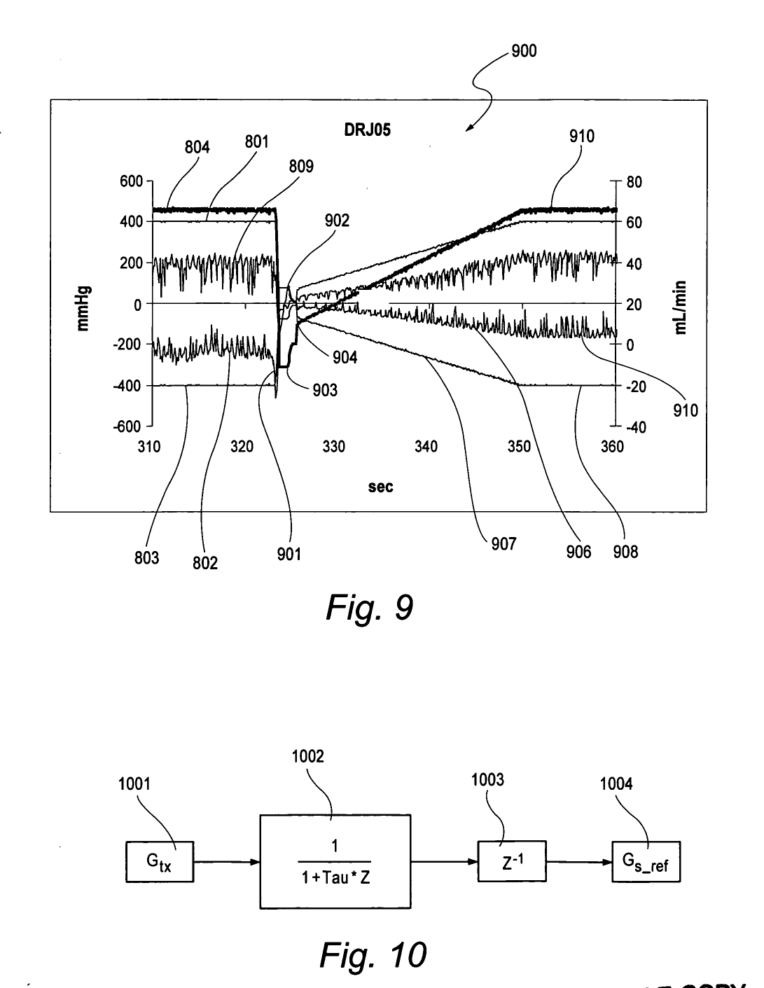

FIG. 10 shows how the reference glucose sensor can be compared directly with the control glucose sensor by modeling the plant between the two sensors. Gtx 101 is first filtered by a low pass filter 1002 that is modeled on the ultrafiltrate volume and ultrafiltrate flow rate. Next the output of the low pass filter 1002 is placed in a delay buffer representing the time delay of the ultrafiltrate to flow from the filter outlet past the reference glucose sensor. This delay is modeled as a function of ultrafiltrate flow and the transit delay between sensors The output of the buffer Gs_ref 1004 is compared directly to the output of the reference glucose sensor. If the signals differ from each other by more than 5 mg/dl for a 5 minute period a control glucose sensor calibration sequence is initiated. This differs from the reference calibration sequence where the ultrafiltrate pump is reversed and the reference calibration signal is calibrated with the glucose calibration solution. The glucose control sensor calibration sequence consists of adjusting the sensitivity of the control glucose sensor until both sensors match.

The preferred embodiment of the invention now known to the invention has been fully described here in sufficient detail such that one of ordinary skill in the art is able to make and use the invention using no more than routine experimentation. The embodiments disclosed herein are not all of the possible embodiments of the invention. Other embodiments of the invention that are within the sprite and scope of the claims are also covered by this patent.

What is claimed is:

1. A method for controlling the level of blood glucose in a patient through an extracorporeal blood circuit, said method comprising:
a. withdrawing blood from a vascular system in the patient to the extracorporeal circuit;
b. removing ultrafiltrate from the withdrawn blood in the circuit;
c. determining a level of glucose present in the blood based on the removed ultrafiltrate;
d. infusing at least a portion of the removed ultrafiltrate and the withdrawn blood into the vascular system, and
e. infusing insulin into the patient based on the determined level of glucose.

2. A method as in claim 1 where the rate of infusion is adjusted based on a current level of glucose and a prior level of glucose.

3. A method as in claim 1 wherein another portion of the removed ultrafiltrate is collected externally of the patient and is not infused.

4. A method as in claim 1 wherein the portion of the removed ultrafiltrate is returned to the withdrawn blood upstream of a filter removing the ultrafiltrate.

5. A method as in claim 1 further comprising introducing a calibration solution into the withdrawn blood and upstream of a filter removing ultrafiltrate, and determining a level of glucose present in the blood based on the removed ultrafiltrate.

6. A method as in claim 1 further comprising introducing a calibration solution into the withdrawn blood and upstream of a filter removing ultrafiltrate and determining a level of glucose present in the blood based on the removed ultrafiltrate, wherein a return of removed ultrafiltrate to the withdrawn blood is interrupted while the calibration solution is introduced into the withdrawn blood.

7. A method as in claim 1 further comprising introducing a calibration solution into the withdrawn blood and upstream of a filter removing ultrafiltrate and determining a level of glucose present in the blood based on the removed ultrafiltrate, wherein the introduction of the calibrated solution is at a controlled rate.

8. A method as in claim 1 wherein the insulin is infused into the patient by first introducing the insulin into the withdrawn blood in the circuit.

9. A method for controlling the level of blood glucose in a patient through an extracorporeal blood circuit, said method comprising:
a. withdrawing blood from a vascular system in the patient to the extracorporeal circuit;
b. controlling a withdrawn blood pressure by adjustment of a blood flow rate through the circuit;
c. removing ultrafiltrate from the blood withdrawn to an ultrafiltrate circuit;
d. using the withdrawn ultrafiltrate to sense a level of glucose present in the blood;
e. infusing at least a portion of the removed ultrafiltrate and the withdrawn blood into the vascular system;
f. controlling a infusion pressure of the infused blood and ultrafiltrate by adjustment of a flow rate of the removed ultrafiltrate and withdrawn blood, and
g. adjusting a rate of infusion of insulin into withdrawn blood based on the sensed level of glucose.

10. A method as in claim 9 wherein the adjustment to the rate of infusion is based on a comparison of a current glucose level measurement to an earlier glucose level measurement.

11. A method as in claim 9 wherein the adjustment of the rate infusion is performed manually in response to an alarm issued when the sensed level of glucose is outside a predetermined glucose range.

12. A method as in claim 9 wherein the portion of the removed ultrafiltrate is returned to the withdrawn blood upstream of a filter removing the ultrafiltrate.

13. A method as in claim 9 further comprising introducing a calibration solution into the withdrawn blood and upstream of a filter removing ultrafiltrate and determining a level of glucose present in the blood based on the removed ultrafiltrate, wherein a return of removed ultrafiltrate to the withdrawn blood is interrupted while the calibration solution is introduced into the withdrawn blood.

14. A system for controlling a blood glucose level in a patient comprising:
a withdrawal blood line in fluid communication with a vascular system of patient;
a filter having an input to receive blood connected to the withdrawal blood line, a blood output, a filter membrane adjacent on one side to a blood passage in the filter, and a ultrafiltrate output on an opposite side of a filter membrane, wherein the ultrafiltrate from the withdrawn blood, passes through the membrane, through the ultrafiltrate output and to an ultrafiltrate line;
a blood glucose sensor monitoring the ultrafiltrate passing through ultrafiltrate line;
an infusion line connected to the blood output of the filter and in fluid communication with the vascular system of the patient;
an ultrafiltrate valve connecting an output of the ultrafiltrate line to the withdrawal blood line, and
an insulin valve connecting an insulin line to said infusion line.

15. The system as in claim 14 wherein the system is an extracorporeal blood circuit connectable to a pump console.

16. The system as in claim 14 wherein the ultrafiltrate valve is a three port valve having a first position to direct ultrafiltrate to the withdrawal blood line and a second position to direct ultrafiltrate to a container.

17. The system as in claim 14 further comprising a calibration fluid valve connecting a calibration fluid line to the ultrafiltrate line.

18. A device for calibrating a glucose sensor in an ultrafiltrate line comprising:
an ultrafiltrate pump connected to an ultrafiltrate line;
a glucose calibration solution source connected to the ultrafiltrate line downstream of the ultrafiltrate pump when the pump is drawing ultrafiltrate from the filter, wherein a one way valve connects the ultrafiltrate line to the ultrafiltrate line to allow the calibration solution to flow out of the calibration solution bag and into the ultrafiltrate line while the pump reverses the flow in the ultrafiltrate line;
a blood glucose sensor connected to said ultrafiltrate line, and
a second one way valve down stream of the ultrafiltrate pump and glucose calibration solution which only allows flow downstream of the ultrafiltrate pump.

19. A method for calibrating a glucose sensor in an ultrafiltrate line connected to an ultrafiltrate pump, said method comprising:
a. periodically reversing the ultrafiltrate pump to withdraw a calibration solution of glucose across the glucose sensor;
b. preventing other fluids from entering the ultrafiltrate line while the pump has reversed the ultrafiltrate flow, and
c. calibrating the glucose sensor to read the same level of glucose as the level of glucose known to be in the calibration solution.
